(19) United States
(12) Patent Application Publication
(10) Pub. No.: US 2021/0217498 A1
(43) Pub. Date: Jul. 15, 2021

(54) DATA PROCESSING APPARATUS AND METHOD FOR PREDICTING EFFECTIVENESS AND SAFETY OF NEW DRUG CANDIDATE SUBSTANCE

(71) Applicant: MEDIRITA, Seoul (KR)

(72) Inventors: Young Woo PAE, Seoul (KR); Seung-Hyun JIN, Seoul (KR)

(73) Assignee: MEDIRITA, Seoul (KR)

(21) Appl. No.: 17/059,417

(22) PCT Filed: Mar. 13, 2019
(86) PCT No.: PCT/KR2019/002920

§ 371 Date: Nov. 27, 2020

(30) Foreign Application Priority Data
Dec. 24, 2018 (KR) 10-2018-0168658
Mar. 13, 2019 (KR) 10-2019-0028790

(51) Int. Cl. G16C 20/10; G16C 20/50; G16C 20/70

(52) U.S. Cl. CPC G16C 20/10; G16C 20/70; G16C 20/50

(57) ABSTRACT

A data processing method for discovering a new drug candidate substance by a data processing apparatus according to an embodiment of the present invention includes receiving a predetermined search word through a user interface unit, extracting a plurality of druggable paths related to the predetermined search word and a druggable path (DP) index for each druggable path by using an artificial neural network (ANN) model, selecting some of the druggable paths having a relatively high DP index among the plurality of druggable paths, extracting information on absorption, distribution, metabolism, excretion, and toxicity (ADMET information) for the some of the druggable paths by using an ADMET model, and outputting the DP index and the ADMET information for each of the some of the druggable paths.

### TECHNICAL FIELD

The present invention relates to a data processing apparatus and method for predicting effectiveness and safety of a new drug candidate substance.

### BACKGROUND ART

It is known that it takes a total of 15 years and costs 2 to 3 trillion won on average to develop a new drug. In the above period of time, it is known that it takes approximately 6 years to discover new drug candidates before preclinical trials.

In general, in order to discover new drug candidates, which is the first step in the pipeline for developing a new drug, a large number of specialized research personnel are going through a process of searching for enormous amounts of information one by one and inferring associations between major biological entities from the search.

According to the Life Intelligence Consortium (2017), which has been recently launched in Japan, it is predicted that when using artificial intelligence technology to develop a new drug, the time taken to develop the new drug may be reduced to about 40%, and the cost may be reduced to about 50% level.

Meanwhile, omics, also known as somatics, is a term that encompasses the entire collection of biological molecules, cells, tissues, organs, and the like, including genomes, and examples thereof include genomics, proteomics, metabolomics, and the like. Recently, the concept of multi-omics, which means a comprehensive and integrated analysis between different levels of omics, has been introduced.

The effectiveness and safety of a new drug are important factors that are to be predicted to be selected as new drug candidate substances. FIG. 1 illustrates a hierarchical structure of the body. In order to develop a new drug with a high goodness of fit and to secure the effectiveness and safety of the new drug, it is required to use the concept of multi-omics that reflects the structural complexity of the human body ranging from a molecular level to the whole body and the relationship of respective stages of presence.

### DISCLOSURE OF THE INVENTION

### Technical Problem

A technical problem to be solved by the present invention is to provide a data processing apparatus and method for discovering a new drug candidate substance.

Another technical problem to be solved by the present invention is to provide a data processing apparatus and method for securing the effectiveness and safety of a new drug through simulations ranging from a molecular level to the whole body.

### Technical Solution

A data processing method for discovering a new drug candidate substance by a data processing apparatus according to an embodiment of the present invention includes: receiving a predetermined search word through a user interface unit; extracting a plurality of druggable paths related to the predetermined search word and a druggable path (DP) index for each druggable path by using an artificial neural network (ANN) model; selecting some of the druggable paths having a relatively high DP index among the plurality of druggable paths; extracting information on absorption, distribution, metabolism, excretion, and toxicity (ADMET information) for the some of the druggable paths by using an ADMET model; and outputting the DP index and the ADMET information for each of the some of the druggable paths.

The data processing method may further include: learning a biological network connecting a plurality of biological entities according to a correlation between the biological entities; and generating the artificial neural network model in advance according to a result of learning the biological network.

A convolution neural network algorithm may be used in the learning, and the result of learning the biological network may be the plurality of druggable paths included in the biological network and the DP index for each druggable path.

The biological network may be a multi-omics network in which some of the plurality of biological entities are included in different omics levels from remaining biological entities thereof.

The multi-omics network may be extracted from a database (DB) matrix including: a DB regarding at least some omics levels selected from among a plurality of omics levels constituting omics through the user interface unit; and a DB regarding at least some types of correlations selected from among a plurality of types of correlations constituting the omics through the user interface unit.

The multi-omics network may connect the plurality of biological entities extracted in relation to the predetermined search word from the DB matrix according to the correlation between the biological entities.

The predetermined search word may be one of a disease name, a compound name, and a drug name.

A data processing apparatus for discovering a new drug candidate substance according to an embodiment of the present invention includes: a user interface unit receiving a predetermined search word; a path selection unit extracting a plurality of druggable paths related to the predetermined search word and a druggable path (DP) index for each druggable path by using an artificial neural network (ANN) model and selecting some of the druggable paths having a relatively high DP index among the plurality of druggable paths; an ADMET information extraction unit extracting information on absorption, distribution, metabolism, excretion, and toxicity (ADMET information) for the some of the druggable paths by using an ADMET model; and an output unit outputting the DP index and the ADMET information for each of the some of the druggable paths.

A recording medium recording a computer-readable program according to an embodiment of the invention causes a computer to perform a data processing method for discovering a new drug candidate substance, the data processing method including: receiving a predetermined search word through a user interface unit; extracting a plurality of druggable paths related to the predetermined search word and a druggable path (DP) index for each druggable path by using an artificial neural network (ANN) model; selecting some of the druggable paths having a relatively high DP index among the plurality of druggable paths; extracting information on absorption, distribution, metabolism, excretion, and toxicity (ADMET information) for the some of the druggable paths by using an ADMET model; and outputting the DP index and the ADMET information for each of the some of the druggable paths.

### Advantageous Effects

According to an embodiment of the present invention, it is possible to significantly reduce the cost and period required to discover a new drug candidate substance with a high goodness of fit.

In particular, according to an embodiment of the present invention, it is possible to obtain an optimal route for a drug to act to guarantee the effectiveness and safety, and also obtain information on the effectiveness and safety for each route.

### BRIEF DESCRIPTION OF THE DRAWINGS

FIG. 1 illustrates a hierarchical structure of the body.

FIG. 2 illustrates a concept of a network.

FIG. 3 is a block diagram of a data processing system for discovering a new drug candidate substance according to an embodiment of the present invention.

FIG. 4 is a flowchart of a data processing method for discovering a new drug candidate substance by a data processing apparatus according to an embodiment of the present invention.

FIGS. 5a to 5c are examples of results output from an output unit of the data processing apparatus according to an embodiment of the present invention.

FIG. 6 is a block diagram of a multi-omics network generation apparatus according to an embodiment of the present invention.

FIG. 7 is a block diagram of an omics DB in the multi-omics network generation apparatus according to an embodiment of the present invention.

FIG. 8 is a flowchart of a method for generating the multi-omics network by the multi-omics network generation apparatus according to an embodiment of the present invention.

FIG. 9 illustrates an example in which an omics level is entered in step S1000 according to an embodiment of the present invention.

FIG. 10 illustrates an example in which a type of correlation is entered in step S1100 according to an embodiment of the present invention.

FIG. 11 illustrates an example of a first matrix generated in step S1300 according to an embodiment of the present invention.

FIG. 12 illustrates an example in which a predetermined search word is entered.

FIG. 13 is a part of an example of a second matrix showing biological entities extracted in step S1500 and a correlation therebetween.

FIG. 14 is an example of a multi-omics network generated according to an embodiment of the present invention.

FIG. 15 is a diagram illustrating a method for generating an ANN model by a model generating apparatus according to an embodiment of the present invention.

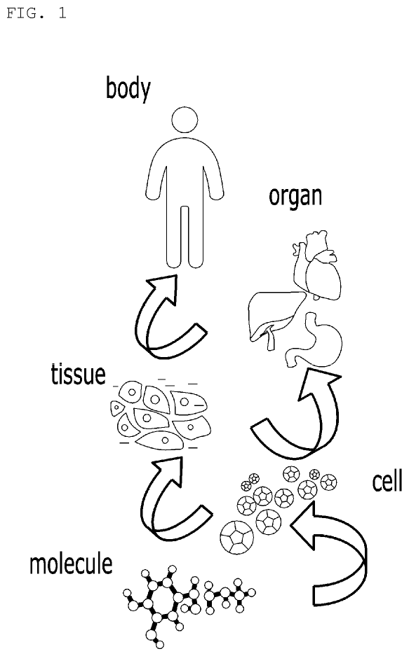

### MODE FOR CARRYING OUT THE INVENTION

It is to be understood that the present invention may be variously modified and embodied, and thus particular embodiments thereof will be illustrated in the drawings and described. However, this is not intended to limit the present invention to the specific embodiments, it should be understood to include all modifications, equivalents, and substitutes included in the spirit and scope of the present invention.

It will be understood that, although the terms second, first, etc. may be used herein to describe various elements, these elements are not limited by these terms. These terms are only used to distinguish one element from another element. For example, without departing from the teachings of the present invention, a second element could be termed a first element, and similarly, a first element could be termed a second element. The term and/or includes a combination of a plurality of related listed items or any of a plurality of related listed items.

It will be understood that when an element is referred to as being “coupled” or “connected” to another element, the element may be directly coupled or connected to the other element, or intervening elements may also be present. In contrast, it will be understood that when an element is referred to as being “directly coupled” or “directly connected” to another element, there are no intervening elements present.

The terms used in the present application are merely provided to describe specific embodiments, and are not intended to limit the present invention. The singular forms, “a”, “an” and “the” are intended to include the plural forms as well, unless the context clearly indicates otherwise. In the present application, it will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms used herein, including technical or scientific terms, have the same meaning as commonly understood by those of ordinary skill in the art to which the embodiments of the present invention belong. Terms, such as those defined in commonly used dictionaries, should be interpreted as having a meaning that is consistent with their meaning in the context of the related art and will not be interpreted in an idealized or overly formal sense unless expressly so defined in the present application.

Hereinafter, embodiments will be described in detail with reference to the accompanying drawings, but identical or corresponding components are denoted by the same reference numerals regardless of figure numbers, and redundant descriptions thereof will be omitted.

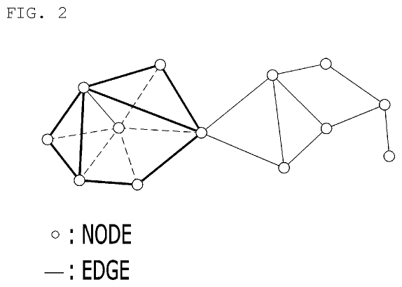

FIG. 2 illustrates a concept of a network.

Referring to FIG. 2, a network may include a plurality of nodes, and two nodes may be connected by an edge. In the present specification, the network may be a knowledge network, a biological network, or a multi-omics network, the node may represent a biological entity, and the edge may represent a correlation between two biological entities.

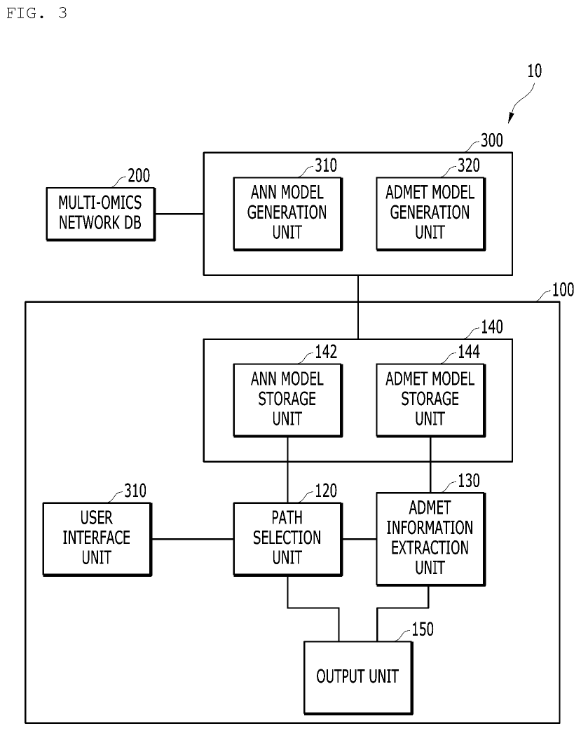

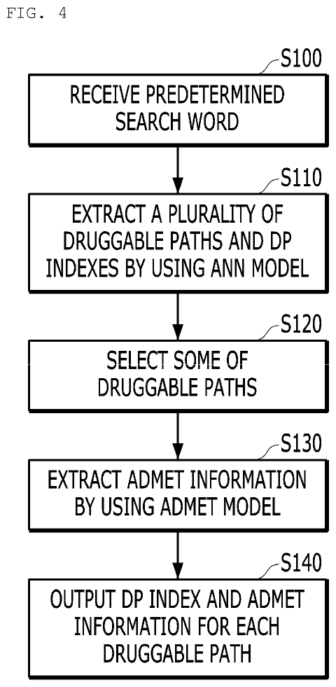

FIG. 3 is a block diagram of a data processing system for discovering a new drug candidate substance according to an embodiment of the present invention, and FIG. 4 is a flowchart of a data processing method for discovering a new drug candidate substance by a data processing apparatus according to an embodiment of the present invention.

Referring to FIG. 3, a data processing system 10 for discovering a new drug candidate substance includes a data processing apparatus 100 that extracts a druggable path and predicts the effectiveness and safety, a multi-omics network DB 200 that stores a multi-omics network in which biological entities belonging to different levels of omics are connected according to the correlation, and a model generation apparatus 300 that extracts the druggable path from the data processing apparatus 100 and generates a model for predicting the effectiveness and safety.

In this case, the data processing apparatus 100 includes a user interface unit 110, a path selection unit 120, an ADMET information extraction unit 130, a storage unit 140, and an output unit 150.

Referring to FIGS. 3 and 4, a predetermined search word, for example, a compound name, a drug name, or a disease name is entered through the user interface unit 110 (S100).

Accordingly, the path selection unit 120 executes an ANN model that is generated in advance and stored in an ANN model storage unit 142 in advance, and extracts a plurality of druggable paths related to the predetermined search word entered in step S100 and DP indexes for each druggable path (S110). Here, the druggable path means a path through which a drug reacts or a path through which a drug acts, and may be used interchangeably with a drug reaction path or a drug action path. In this case, the druggable path may be displayed according to a correlation between biological entities in different omics levels, and may be some paths in a multi-omics network extracted by a predetermined search word to be described later in the present specification. In addition, the DP index for each druggable path may be an index indicating the degree to which a path is predicted to be suitable as a druggable path, and the higher DP index, the more suitable druggable path may be. In this case, the DP index may be a probability value.

Next, the path selection unit 120 selects some druggable paths having a relatively high DP index among the plurality of druggable paths extracted in step S110 (S120). Here, the number of selected druggable paths may be preset by a user or may be preset by software.

Next, the ADMET information extraction unit 130 extracts ADMET information by executing an ADMET model that is generated in advance and stored in the ADMET model storage unit 144 in advance for some of the druggable paths selected in step S120 (S130). Here, the ADMET information may be information indicating the effectiveness and safety for a predetermined compound, and may include a plurality of indicators indicating at least some of absorption, distribution, metabolism, excretion, and toxicity. Since the ADMET information is an indicator for each compound, the same ADMET information may be extracted when the compounds included in the druggable path are the same, even if the DP index is different.

Next, the output unit 150 outputs DP index and ADMET information for each of some druggable paths extracted in step S120 in relation to the predetermined search word (S140).

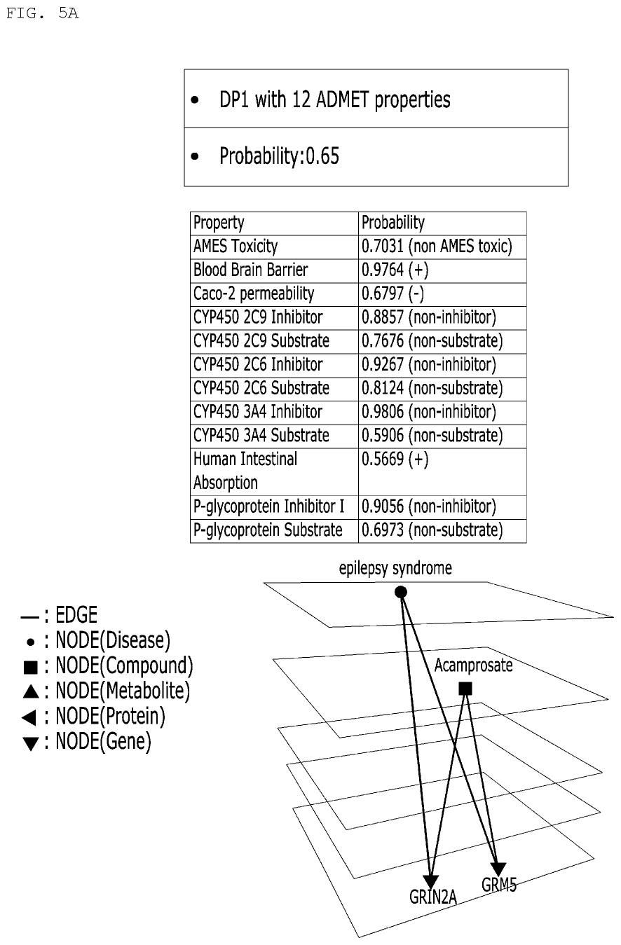

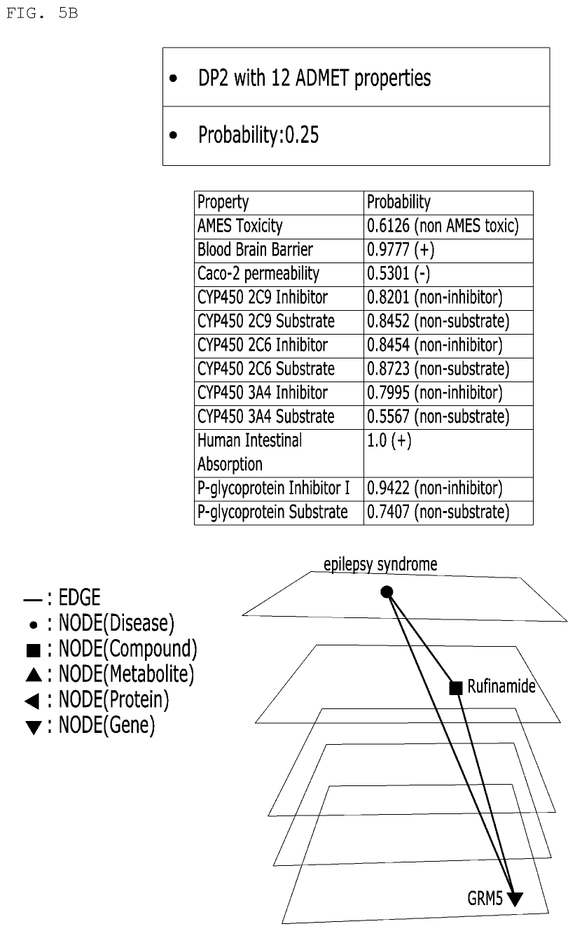

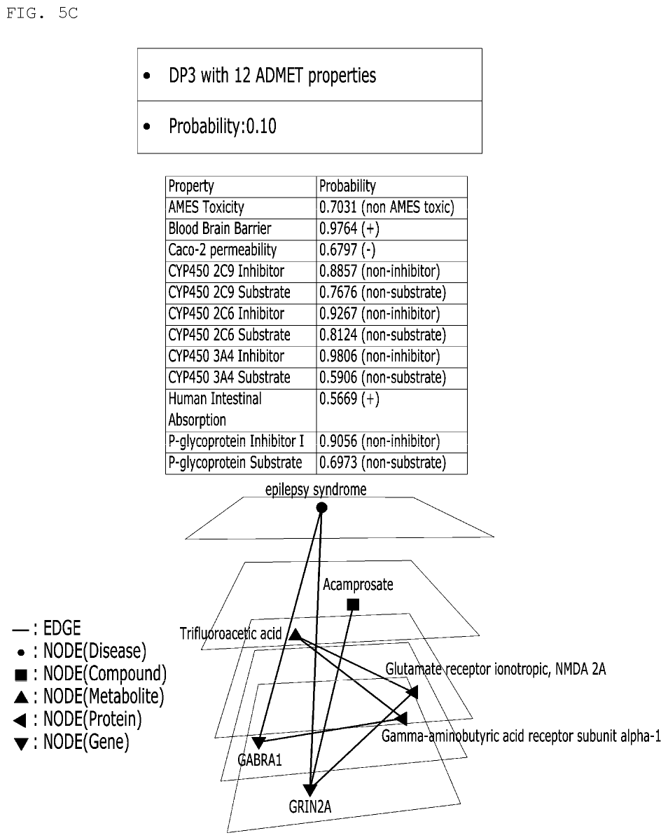

FIGS. 5a to 5c are examples of results output from an output unit of the data processing apparatus according to an embodiment of the present invention. For example, when a disease name “epilepsy syndrome” is entered as the search word, in step S120, GRIN2A, GRM5, and acamprosate may be biological entities, DP1, GRM5, and rufinamide, which have a DP index of 0.65, may be biological entities, DP2, GRIN2A, GABRA1, and acamprosate, which have a DP index of 0.25, may be biological entities, and DP3, which has a DP index of 0.1, may be selected as a druggable path. Accordingly, in step S130, ADMET information on acamprosate, which is a compound included in DP1, ADMET information on rufinamide, which is a compound included in DP2, and ADMET information on acamprosate, which is a compound included in DP3, are extracted, and the druggable paths, the DP indexes, and ADMET indexes may be exposed for each druggable path as illustrated in FIGS. 5a to 5c. In this case, the ADMET information may include a plurality of indicators representing at least some of absorption, distribution, metabolism, excretion and toxicity as described above, and here, twelve indicators such as “AMES Toxicity”, “Blood Brain Barrier”, “Caco-2 permeability”, “CYP450 2C9 inhibitor”, “CYP450 2C9 substrate”, “CYP450 2D6 inhibitor”, “CYP450 2D6 substrate”, “CYP450 3A4 inhibitor”, “CYP450 3A4 substrate”, “Human Intestinal”, “Absorption”, “P-glycoprotein inhibitor” and “P-glycoprotein substrate” are expressed as probability values; however, these are exemplary and the ADMET information is not limited thereto.

Meanwhile, according to an embodiment of the present invention, in order for the data processing apparatus 100 to extract the druggable path and the DP index for the predetermined search word, and to extract ADMET information, an ANN model and an ADMET model may be generated in advance.

Here, a model generation apparatus 300 including an ANN model generation unit 310 and an ADMET model generation unit 320 is illustrated as a separate configuration disposed outside the data processing apparatus 100, but the present invention is limited thereto. At least one of the ANN model generation unit 310 and the ADMET model generation unit 320 may be included in the data processing apparatus 100.

The ANN model generation unit 310 and the ADMET model generation unit 320 may use the multi-omics network DB 200 to generate the ANN model and the ADMET model. Hereinafter, a method for generating the multi-omics network DB 200 will be first described in detail, and then a method for generating the ANN model and the ADMET model by using the multi-omics network DB 200 will be described.

First, the multi-omics network DB 200 may be a DB constructed by the multi-omics network generated in advance in relation to various search words. The multi-omics network refers to a network in which a plurality of nodes including a plurality of biological entities are connected according to the correlation between the plurality of biological entities, and the method for generating the multi-omics network may be described as follows.

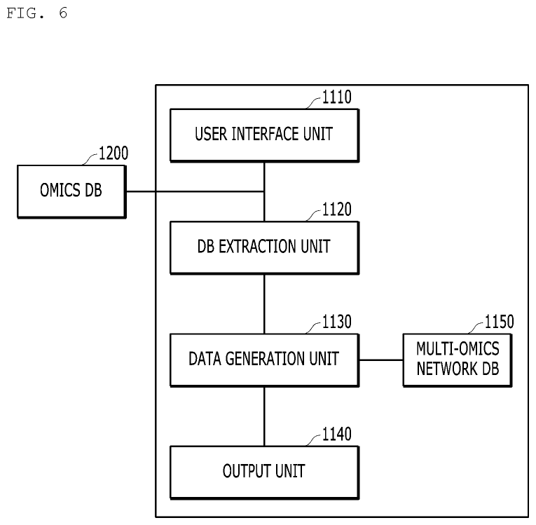

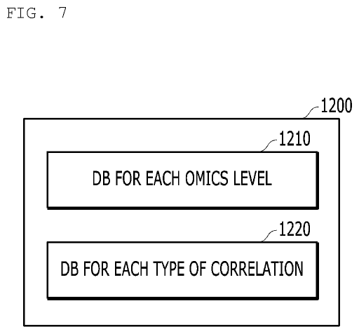

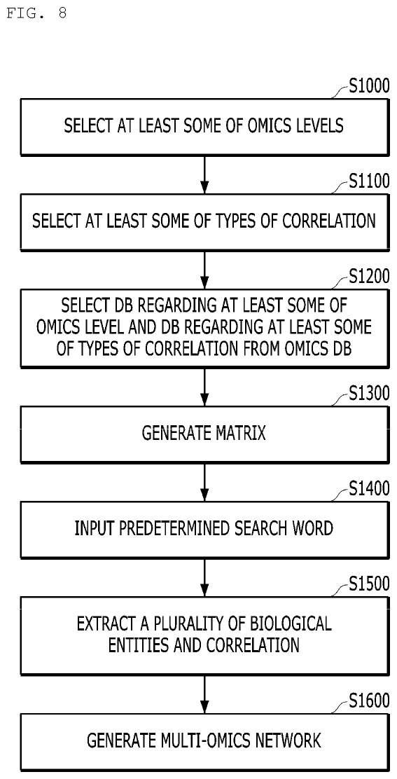

FIG. 6 is a block diagram of a multi-omics network generation apparatus according to an embodiment of the present invention, FIG. 7 is a block diagram of an omics DB in the multi-omics network generation apparatus according to an embodiment of the present invention, and FIG. 8 is a flowchart of the method for generating the multi-omics network by the multi-omics network generation apparatus according to an embodiment of the present invention.

Referring to FIG. 6, a multi-omics network generation device 1100 includes a user interface unit 1110, a DB extraction unit 1120, a data generation unit 1130, a data output unit 1140, and a multi-omics network DB 1150.

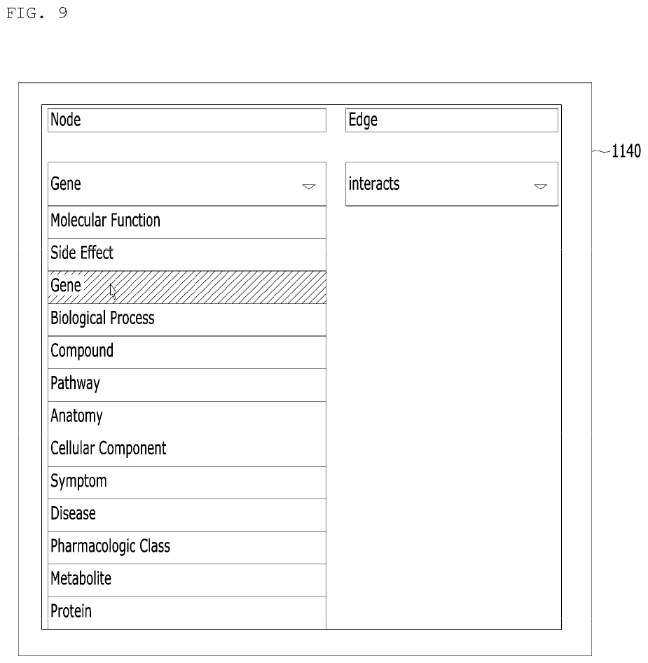

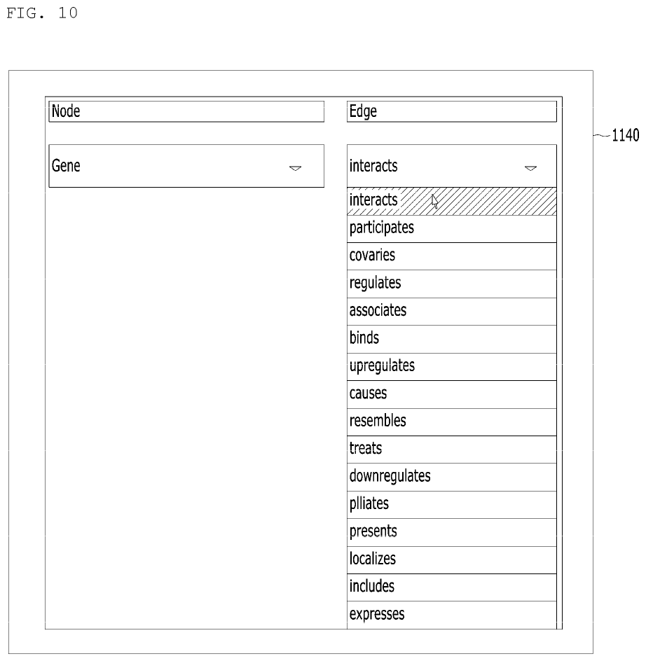

Referring to FIGS. 6 to 8, the user interface unit 1110 receives at least some of the omics levels among a plurality of levels constituting omics (S1000), and receives at least some types of correlations among a plurality of types of correlation constituting the omics (S1100). Here, omics, also known as somatics, may include, for example, genetics, transcriptomes, proteomics, metabolomics, epigenetics, geology, and the like, and may include details related to anatomy, biological processes, pathways, pharmacological classes, symptoms, diseases, compounds, drugs, side effects, and the like, but is not limited thereto. A plurality of omics levels may include a gene level, a transcription level, a protein level, a metabolite level, an epigene level, a lipid level, an anatomical structure level, a biological process level, a pathway level, a pharmacologic class level, a symptom level, a disease level, a compound level, a drug level, a side effect level, and the like, but are not limited thereto. Here, the anatomical structure may mean a tissue, an organ, or the like, the biological process may be a series of events that include cellular components, such as locations at the level of the structure in cell, and molecular functions extracted from gene ontology, and the pharmacological class may be a pharmacological effect, a mechanism of action. The plurality of types of correlation may include “interact”, “participate”, “covariate”, “regulate”, “associate”, “bind”, “upregulate”, “cause”, “resemble”, “treat”, “downregulate”, “palliate”, “present”, “localize”, “include”, and “express”, and any identification numbers or identification symbols may for each type be assigned. The identification number or identification symbol for each type may be set by the user or may be set automatically. FIG. 9 illustrates an example in which an omics level is entered in step S1000 according to an embodiment of the present invention, and FIG. 10 illustrates an example in which a type of correlation is entered in step S1100 according to an embodiment of the present invention. Referring to FIG. 9, a screen in which a plurality of omics levels may be selected may be exposed through the output unit 1140, and at least some of the omics levels may be selected from among the plurality of omics levels through the user interface unit 1110. Further, referring to FIG. 10, a screen in which a plurality of types of correlation may be selected may be exposed through the output unit 1140, and at least some of the types of correlations may be selected from among the plurality of types of correlation through the user interface unit 1110.

Next, the DB extraction unit 1120 extracts a DB regarding at least some of the omics levels selected in step S1000 and a DB regarding at least some of the types of correlations selected in step S1100 from the omics DB (S1200). Here, the omics DB 1200 may be a big data DB, may be a DB outside the multi-omics network generation device 1100 according to an embodiment of the present invention, and may be a global public DB that is accessible by anyone or accessible by a person who has been authenticated under predetermined conditions. The omics DB 1200 may store information on the omics level and information on the correlation between biological entities within the omics level in advance. For example, as illustrated in FIG. 7, the omics DB 1200 may include a DB 1210 for each omics level and a DB 1220 for each type of correlation. For example, the DB 1210 for each omics level is a gene DB, a transcription DB, a protein DB, a metabolite DB, an epigene DB, a lipid DB, an anatomical structure DB, a biological process DB, a pathway DB, a symptom DB, a disease DB, a compound DB, a drug DB, and a side effect DB. In addition, the DB 1220 for each type of correlation may include an interact DB, a participate DB, a covariate DB, a regulate DB, an associate DB, a bind DB, an upregulate DB, a cause DB, a resemble DB, a treat DB, a downregulate DB, a palliate DB, a present DB, a localize DB, an include DB, and an express DB. The DBs may be managed and operated by being integrated into one big data DB, or may be managed and operated in a distributed manner.

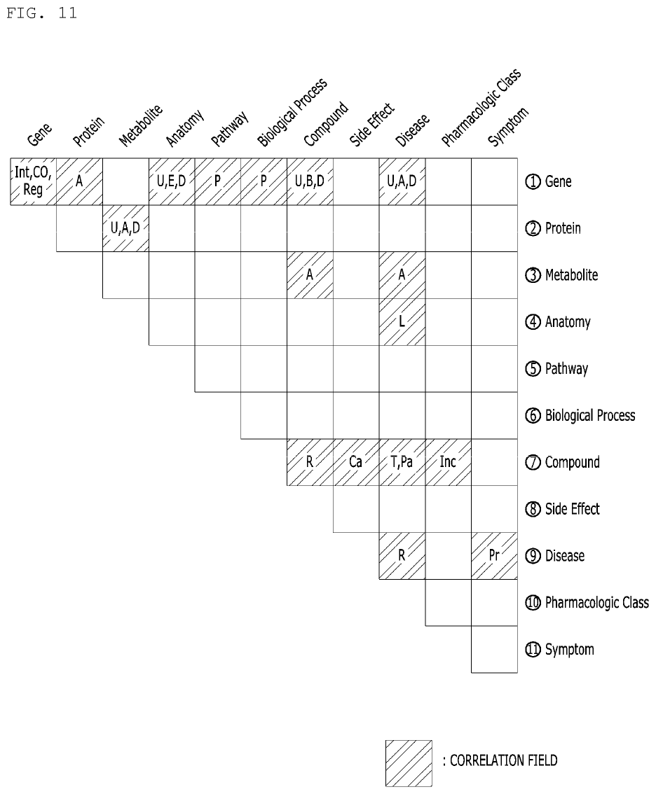

In addition, the DB extraction unit 1120 generates a first matrix including a DB regarding at least some of the omics levels extracted in step S1200 and a DB regarding at least some of the types of correlations (S1300). Here, the first matrix may be referred to as a set of DBs extracted in step S1200. FIG. 11 illustrates an example of the first matrix generated in step S1300 according to an embodiment of the present invention. Referring to FIG. 11, the omics levels selected in step S1000 are arranged on each of the horizontal and vertical axes, and the types of correlation selected in step S1100 may be generated to be displayed at points where the horizontal and vertical axes intersect. For example, a gene level, a protein level, a metabolite level, an anatomical structure level, a pathway level, a biological process level, a compound level, a side effect level, a disease level, a pharmacological class level and a symptom level may be arranged on each of the horizontal and vertical axes of the first matrix, and at points where the horizontal and vertical axes intersect, at least one of interact (Int), participate (P), covariate (Co), regulate (Reg), associate (A), bind (B), upregulate (U), cause (Ca), resemble (R), treat (T), downregulate (D), palliate (Pa), present (Pr), localize (L), include (Inc), and express (E), which are types of correlations, may be displayed.

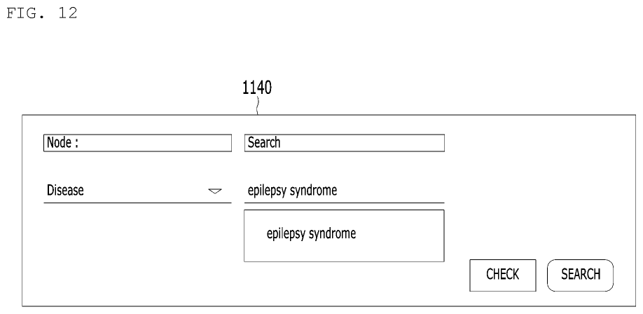

Meanwhile, the user interface unit 1110 receives a predetermined search word (S1400). The predetermined search word may be a search word to be used when a user would like to search for information, and may be one of a plurality of biological entities included for each ohmic level, for example, one of a gene name, a protein name, a metabolite name, a symptom name, a disease name, a compound name, a drug name, or a side effect name. FIG. 12 illustrates an example in which a predetermined search word is entered. Referring to FIG. 12, a screen for entering a predetermined search word may be exposed through the output unit 1140, and the predetermined search word may be entered through the user interface unit 1110. In the example of FIG. 12, a disease name is selected as a category and an epilepsy syndrome is entered as a predetermined search word.

Next, the data generation unit 1130 extracts at least one biological entity related to the predetermined search word received in step S1400 by using the first matrix generated in step S1300, and extracts the correlation between the predetermined search word and the extracted biological entity by using the first matrix generated in step S1300 (S1500). Here, the biological entity may include at least one of the gene, the protein, the metabolite, the symptom, the disease, the compound, and the drug, and the omics level to which the predetermined search word belongs may be the same as or difficult from the ohmic level to which the biological entity belongs. For example, as illustrated in FIG. 12, when the predetermined search word is the epilepsy syndrome, which is a disease name, the biological entity extracted in step S1500 may include at least one of a gene associated with the epilepsy syndrome, a protein associated with the epilepsy syndrome, a metabolite associated with the epilepsy syndrome, a symptom associated with the epilepsy syndrome, a disease associated with the epilepsy syndrome, a compound associated with the epilepsy syndrome, and a drug associated with the epilepsy syndrome. To this end, the data generation unit 1130 may extract the biological entities associated with the epilepsy syndrome from each of the gene DB, the protein DB, the metabolite DB, the anatomical structure DB, the pathway DB, the biological path DB, the compound DB, the side effect DB, the disease DB, the pharmacological class DB, and symptom DB, which constitute the first matrix in step S1300. Accordingly, the biological entity extracted in step S1500 may include at least one of a plurality of genes associated with the epilepsy syndrome, a plurality of proteins associated with the epilepsy syndrome, a plurality of metabolites associated with the epilepsy syndrome, a plurality of symptoms associated with the epilepsy syndrome, a plurality of diseases associated with the epilepsy syndrome, a plurality of compounds associated with the epilepsy syndrome, and a plurality of drugs associated with the epilepsy syndrome.

As described above, when the biological entities and correlations associated with the predetermined search word are extracted by using the first matrix in step S1300, it is possible to significantly reduce the amount of DB to be searched, and accordingly, it is possible to reduce time and cost for searching for information, and it is possible to extract only the information desired by the user.

In this case, in order for the data generation unit 1130 to extract at least one biological entity related to the predetermined search word and the correlation between the biological entities, the data generation unit 1130 may use a natural language processing algorithm based on artificial intelligence technology including machine learning. Here, the natural language processing refers to all kinds of technologies for mechanically analyzing language phenomena spoken by humans to make them into a form that is to be understood by a computer, and express the form that is to be understood by the computer in language that is to be understood by humans. To this end, the omics DB 1200 may be a language-based DB for each biological entity type, and may include information reflecting a machine learning result and a feedback result.

Alternatively, in order for the data generation unit 1130 to extract at least one biological entity related to the predetermined search word and the correlation between the biological entities, the data generation unit 1130 may also use a deep natural network algorithm based on artificial intelligence technology including machine learning. Here, the deep neural network is an ANN including several hidden layers between an input layer and an output layer, and refers to all kinds of technologies used for classification, prediction, image recognition, character recognition, or the like. To this end, the omics DB 1200 may be an image-based DB for each biological entity type, and may include information reflecting a machine learning result and a feedback result.

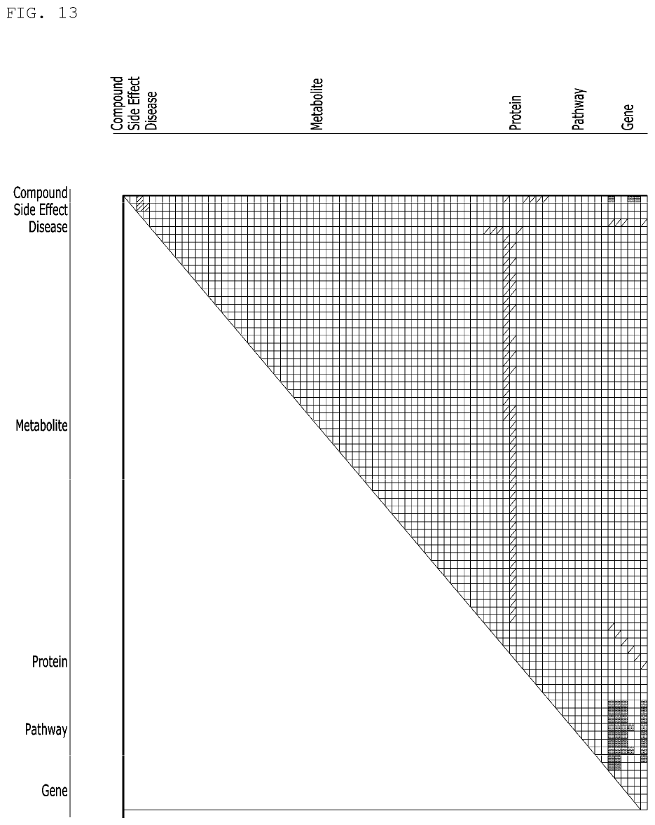

FIG. 13 is a part of an example of a second matrix showing biological entities extracted in step S1500 and a correlation therebetween. Referring to FIG. 13, the second matrix may be generated such that a plurality of biological entities are sequentially disposed on each of the horizontal and vertical axes according to the hierarchical structure of the omics levels, and correlations between the plurality of biological entities are displayed at points where the horizontal and vertical axes intersect. For example, when the ohmic levels selected in step S1000 are the gene level, the pathway level, the protein level, the metabolite level, the disease level, the side effect level, and the compound level, and the predetermined search word entered in step S1400 is bupropion, which is one of the compounds, it can be seen that in step S1500, a plurality of genes, a plurality of pathways, a plurality of proteins, a plurality of metabolites, a plurality of diseases, a plurality of side effects, and a plurality of compounds, which are associated with bupropion, are extracted as biological entities, and the biological entities are sequentially disposed on each of the horizontal and vertical axes according to the hierarchical structure of the ohmic levels. In addition, it can be seen that the correlations between biological entities are displayed in different colors at the points where the horizontal and vertical axes intersect.

The shape of the second matrix is exemplary, and is not limited thereto, and may be modified in various shapes.

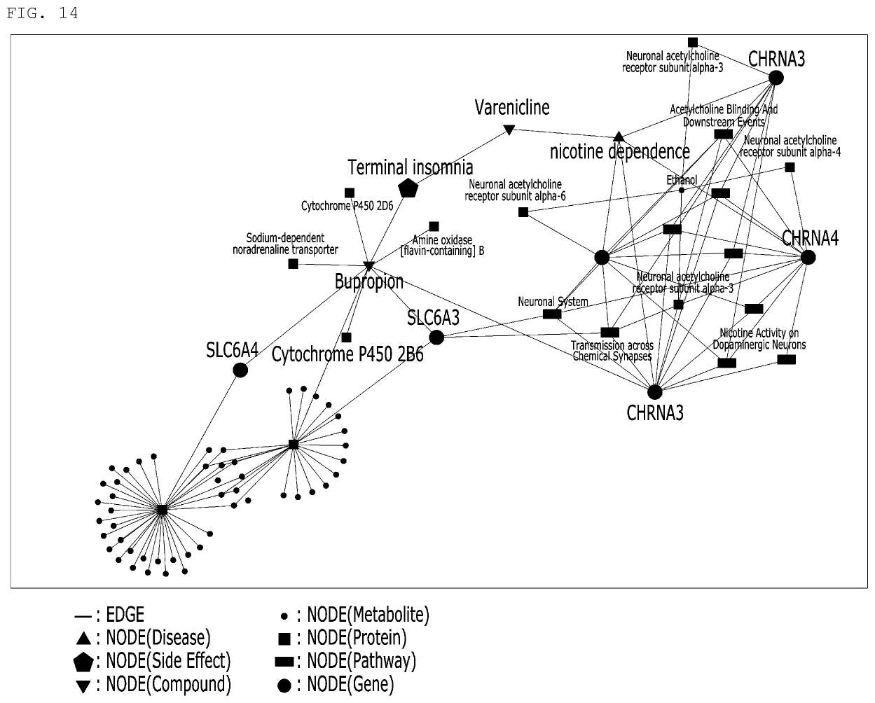

Next, the data generation unit 1130 generates the multi-omics network by using the result extracted in step S1500 (S1600). FIG. 14 is an example of the multi-omics network generated according to an embodiment of the present invention. Here, the multi-omics network may include, as nodes, the predetermined search word received in step S1400 and the biological entities extracted in step S1500, and may be in a form in which a plurality of nodes are connected by using connection lines according to the correlation between the predetermined search word and the biological entity, which are extracted in step S1500, or the correlation between the biological entities. There are various paths from node A, which is one of the nodes in the multi-omics network, to node B, which is the other, and all possible paths may be connected by connection lines. Here, the multi-omics network is a network constituted by the correlations between biological entities, and may be interchangeable with the biological network. In the multi-omics network, some of the plurality of biological entities that may be nodes may be included in a different omics level from the other biological entities. That is, as illustrated in FIG. 14, the multi-omics network may include, as nodes, a plurality of biological entities included in different ohmic levels such as the gene level, the pathway level, the protein level, the metabolite level, the compound level, the side effect level, and the disease level, and some of a plurality of biological entities included in the gene level may be connected to some of a plurality of biological entities included in the protein level, or connected to some of a plurality of biological entities included in the pathway level. Likewise, some of a plurality of biological entities included in the compound level may be connected to some of the plurality of biological entities included in the protein level, connected to some of the plurality of biological entities included in the pathway level, or connected to some of a plurality of biological entities included in the side effect level.

As described above, according to an embodiment of the present invention, when some of the plurality of omics levels and some of the plurality of types of correlations are received through the user interface unit 1110, the DB regarding the corresponding omics levels and the DB regarding the types of correlation are automatically extracted, which makes it possible to significantly reduce the amount of information to be searched by the multi-omics network generation device 1100 and accordingly possible to obtain the multi-omics network including the omics levels and the types of correlation desired by the user. In addition, according to an embodiment of the present invention, when some of the plurality of omics levels and some of the plurality of types of correlations are received through the user interface unit 1110, it is possible to obtain the multi-omics network including the omics levels and the types of correlations desired by the user, and accordingly possible to easily grasp the hierarchical structure of a plurality of biological entities associated with the predetermined search word within the omics levels desired by the user.

The multi-omics network generated according to the above method may be stored, and when multiple multi-omics networks are stored, the multi-omics network DB 1150 may be constructed.

Here, the multi-omics network DB 1150 is illustrated as being a part of the multi-omics network generation device 1100, but is not limited thereto, and the multi-omics network DB 1150 may be an external configuration of the multi-omics network generation device 1100. That is, the multi-omics network DB 1150 of FIG. 6 may be the multi-omics network DB 200 of FIG. 3. Alternatively, a plurality of multi-omics network DBs 1150 of FIG. 6 may be gathered to construct the multi-omics network DB 200 of FIG. 3.

Next, the model generation apparatus 300 generates the ANN model by using the multi-omics network DB constructed in the above method.

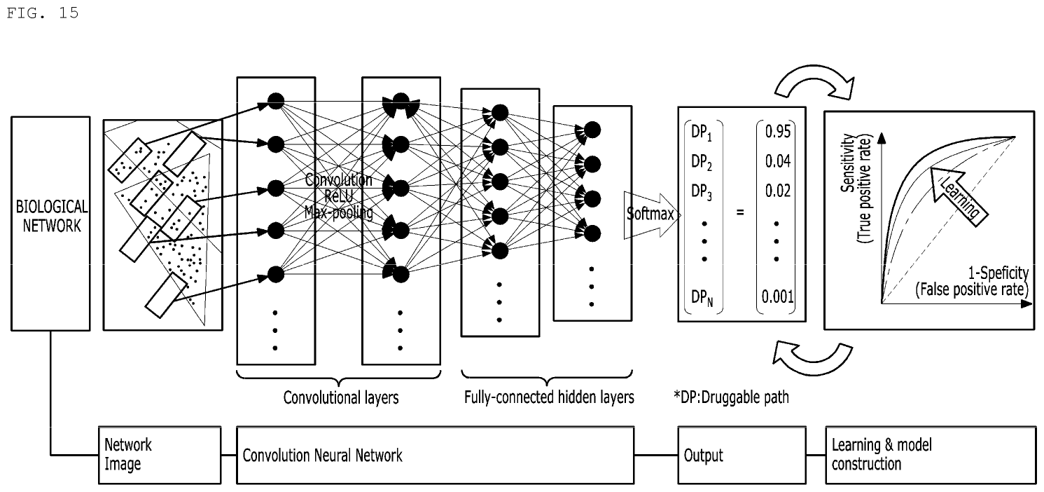

FIG. 15 is a diagram illustrating a method for generating the ANN model by the model generation apparatus according to an embodiment of the present invention.

Referring to FIG. 15, the model generation apparatus 300 may generate the ANN model by learning the multi-omics network stored in the multi-omics network DB 200. To this end, the ANN model generation unit 310 may use a convolution neural network (CNN) algorithm, and the result of the ANN model generation unit 310 may be a plurality of druggable paths included in each biological network and a DP index for each druggable path.

More specifically, the multi-omics network stored in the multi-omics network DB 200 may be entered to the ANN model generation unit 310. In this case, the multi-omics network may be entered in the form of a plurality of divided images, and the plurality of divided images may be calculated through a convolution neural network algorithm. That is, the plurality of divided images may be output in the form of the DP index for each druggable path after calculation and soft-max processes using a convolutional layer and a fully-connected hidden layer. In addition, the DP index for each druggable path may be optimized by repeating a process of learning sensitivity and specificity with a pre-learned training set. To this end, a plurality of druggable paths or a plurality of divided images in the multi-omics network may be tagged in advance.

Likewise, the model generation apparatus 300 may extract ADMET information for each compound from the multi-omics network DB 200 or the omics DB 1200, and may learn the ADMET information to generate an ADMET model. Here, the multi-omics network DB 200 or the omics DB 1200 may include at least one of a compound DB and a drug DB. Alternatively, the ADMET model may be generated using a known modeling technique, for example, a known method in “Wang et al., 2015. In silico ADME/T modeling for rational drug design, Quarterly Reviews of Biophysics”; however, it is exemplary and the ADMET model is not limited thereto.

As described above, according to an embodiment of the present invention, it is possible to generate the ANN model and the ADMET model by using the multi-omics network that reflects the structural complexity of the human body and the relationship for each expression stage, and to extract the druggable path and the ADMET information for the predetermined search word by using the ANN model and ADMET model. Accordingly, it is possible to obtain the effect of whole body simulation, and it is possible to easily obtain the effectiveness and safety in consideration of the hierarchical structure of the human body for a new drug candidate substance.

The term ‘unit’ used in this embodiment refers to software component or hardware components such as a field-programmable gate array (FPGA) or an application specific integrated circuit (ASIC), and ‘unit’ performs certain functions. However, ‘unit’ may not be limited to software or hardware components. ‘unit’ may be configured to be in an addressable storage medium, or may be configured to reproduce one or more processors. Therefore, for example, ‘unit’ may include components such as software components, object-oriented software components, class components, and task components, and may include processors, functions, attributes, procedures, sub-routines, segments of program code, drivers, firmware, micro codes, circuits, data, a database, data structures, tables, arrays, and variables. Functions provided in the components and the ‘unit’ may be coupled with lesser numbers of components and ‘units’, or may be further divided into additional components and ‘units’. Furthermore, the components and ‘units’ may be implemented to reproduce one or more CPUs in a device or a security multimedia card.

Although the embodiments of the present invention have been described above, it is understood that one ordinary skilled in the art can make various changes and modifications to the present invention without departing from the spirit and scope of the present invention as hereinafter claimed.

1. A data processing method for discovering a new drug candidate substance by a data processing apparatus, the data processing method comprising:
receiving a predetermined search word through a user interface unit;
extracting a plurality of druggable paths related to the predetermined search word and a druggable path (DP) index for each druggable path by using an artificial neural network (ANN) model;
selecting some of the druggable paths having a relatively high DP index among the plurality of druggable paths;
extracting information on absorption, distribution, metabolism, excretion, and toxicity (ADMET information) for the some of the druggable paths by using an ADMET model; and
outputting the DP index and the ADMET information for each of the some of the druggable paths.

2. The data processing method of claim 1, further comprising:
learning a biological network connecting a plurality of biological entities according to a correlation between the biological entities; and
generating the artificial neural network model in advance according to a result of learning the biological network.

3. The data processing method of claim 2, wherein a convolution neural network algorithm is used in the learning, and
the result of learning the biological network is the plurality of druggable paths included in the biological network and the DP index for each druggable path.

4. The data processing method of claim 3, wherein the biological network is a multi-omics network in which some of the plurality of biological entities are included in different omics levels from remaining biological entities thereof.

5. The data processing method of claim 4, wherein the multi-omics network is extracted from a database (DB) matrix including:
a DB regarding at least some omics levels selected from among a plurality of omics levels constituting omics through the user interface unit; and
a DB regarding at least some of types of correlations selected from among a plurality of types of correlations constituting the omics through the user interface unit.

6. The data processing method of claim 5, wherein the multi-omics network connects the plurality of biological entities extracted in relation to the predetermined search word from the DB matrix according to the correlation between the biological entities.

7. The data processing method of claim 1, wherein the predetermined search word is one of a disease name, a compound name, and a drug name.

8. A data processing apparatus for discovering a new drug candidate substance, the data processing apparatus comprising:
a user interface unit receiving a predetermined search word;
a path selection unit extracting a plurality of druggable paths related to the predetermined search word and a druggable path (DP) index for each druggable path by using an artificial neural network (ANN) model and selecting some of the druggable paths having a relatively high DP index among the plurality of druggable paths;
an ADMET information extraction unit extracting information on absorption, distribution, metabolism, excretion, and toxicity (ADMET information) for the some of the druggable paths by using an ADMET model; and
an output unit outputting the DP index and the ADMET information for each of the some of the druggable paths.

9. The data processing apparatus of claim 8, further comprising:
a storage unit storing the artificial neural network model,
wherein the artificial neural network model is generated in advance according to a result of learning a biological network connecting a plurality of biological entities according to a correlation between the biological entities.

10. The data processing apparatus of claim 9, further comprising a generation unit generating the artificial neural network model,
wherein the generation unit uses a convolution neural network algorithm to learn the biological network connecting the plurality of biological entities according to the correlation between the biological entities, and
the result of learning the biological network is the plurality of druggable paths included in the biological network and the DP index for each druggable path.

11. The data processing apparatus of claim 10, wherein the biological network is a multi-omics network in which some of the plurality of biological entities are included in different omics levels from remaining biological entities thereof.

12. The data processing apparatus of claim 11, wherein the multi-omics network is extracted from a DB matrix including:
a DB regarding at least some omics levels selected from among a plurality of omics levels constituting omics through the user interface unit; and
a DB regarding at least some types of correlations selected from among a plurality of types of correlations constituting the omics through the user interface unit.

13. The data processing apparatus of claim 12, wherein the multi-omics network connects the plurality of biological entities extracted in relation to the predetermined search word from the DB matrix according to the correlation between the biological entities.

14. The data processing apparatus of claim 8, wherein the predetermined search word is one of a disease name, a compound name, and a drug name.

15. A recording medium having recorded thereon a computer-readable program for causing a computer to perform a data processing method for discovering a new drug candidate substance, the data processing method comprising:
receiving a predetermined search word through a user interface unit;
extracting a plurality of druggable paths related to the predetermined search word and a druggable path (DP) index for each druggable path by using an artificial neural network (ANN) model;
selecting some of the druggable paths having a relatively high DP index among the plurality of druggable paths;
extracting information on absorption, distribution, metabolism, excretion, and toxicity (ADMET information) for the some of the druggable paths by using an ADMET model; and
outputting the DP index and the ADMET information for each of the some of the druggable paths.
